(19) United States
(12) Patent Application Publication
(10) Pub. No.: US 2025/0334584 A1
(43) Pub. Date: Oct. 30, 2025

(54) Method, Controller, and Analyzer for Classifying Microorganism

(71) Applicants: SHIMADZU CORPORATION, Kyoto-shi (JP); OSAKA INSTITUTE OF PUBLIC HEALTH, Osaka-shi (JP)

(72) Inventors: Koichi KOJIMA, Kyoto-shi (JP); Yuki WAKABAYASHI, Osaka-shi (JP); Shunya NISHIJIMA, Osaka-shi (JP); Junko SAKATA, Osaka-shi (JP)

(21) Appl. No.: 18/880,054

(22) PCT Filed: Jun. 29, 2023
(86) PCT No.: PCT/JP2023/024115

§ 371 Date: Dec. 30, 2024

(30) Foreign Application Priority Data
Jun. 30, 2022 (JP) 2022-106329

(51) Int. Cl. G01N 33/68; C12N 1/20

(52) U.S. Cl. CPC G01N 33/6851; C12N 1/205; G01N 2333/24

(57) ABSTRACT

A method for classifying a microorganism, including (S1) obtaining a first mass spectrum resulting from mass spectrometry of a first microorganism belonging to the order Enterobacterales that has been cultured under conditions for producing an acid shock protein, classification of the first microorganism at and below one of the family, genus, and species levels being unknown; (S2) obtaining a first m/z corresponding to the acid shock protein from the first mass spectrum; and (S3) performing classification at or below one of the unknown levels of the first microorganism by analyzing the first m/z.

### TECHNICAL FIELD

The present invention relates to a method, a controller, and an analyzer for classifying a microorganism.

### BACKGROUND ART

Microorganisms are classified at levels of the family, the genus, the species, the strain, and the like, and are known to have properties in accordance with the classification. It is also known that a microorganism can cause various diseases by causing environmental change in accordance with the properties thereof. WO2020/202861 (PTL 1) describes that pathogenicity and drug resistance are different among microorganisms of different strains. Therefore, it is important in the field of study on microorganism and in medical settings to appropriately classify a microorganism, and to grasp the properties thereof by the classification.

### CITATION LIST

### Patent Literature

PTL 1: WO2020/202861

### Non Patent Literature

NPL 1: National Institute of Infectious Diseases, “Escherichia no shinshu, E. albertii, ni tsuite (New Strain of Escherichia, E. albertii)” (IASR Vo. 33, p. 134-136: 2012 May issue)

### SUMMARY OF INVENTION

### Technical Problem

For example, some of Escherichia coli (hereinafter referred to also as “E. coli”) belonging to the order Enterobacterales have a gene producing verotoxin, and cause enterohemorrhagic Escherichia coli infection. Enterohemorrhagic Escherichia coli infection may cause serious symptoms such as hemolytic uremic syndrome in some cases. On the other hand, NPL 1 describes that some of Escherichia albertii (hereinafter also referred to as “E. albertii”) that is a related species of E. coli also have a verotoxin gene, and may be misidentified as enterohemorrhagic Escherichia coli. As described in NPL 1, however, there are only a few properties that allow easy distinction of E. albertii from other bacterial species, and it has been difficult to distinguish E. albertii from E. coli based on biochemical characteristics.

The present disclosure has been devised to solve this problem, and an object is to classify a microorganism belonging to the order Enterobacterales by a simple method.

### Solution to Problem

A method for classifying a microorganism according to a first aspect of the present disclosure is a method for classifying a microorganism, including: obtaining a first mass spectrum resulting from mass spectrometry of a first microorganism belonging to the order Enterobacterales that has been cultured under conditions for producing an acid shock protein, classification of the first microorganism at and below one of the family, genus, and species levels being unknown; obtaining a first m/z corresponding to the acid shock protein from the first mass spectrum; and performing classification at or below one of the unknown levels of the first microorganism by analyzing the first m/z.

### Advantageous Effects of Invention

According to a method for classifying a microorganism of the present disclosure, a first organism can be classified at or below one of unknown levels. Therefore, a microorganism belonging to the order Enterobacterales can be classified by a simple method.

### BRIEF DESCRIPTION OF DRAWINGS

FIG. 1 is a schematic diagram illustrating the configuration of an analyzer according to an embodiment.

FIG. 2 is a flowchart illustrating processing for classifying a microorganism according to the embodiment.

FIG. 3 is a flowchart illustrating processing for obtaining a first m/z.

FIG. 4 is a flowchart illustrating processing for classifying a microorganism according to Embodiment 1.

FIG. 5 is a flowchart illustrating processing for obtaining a second m/z.

FIG. 6 is a flowchart illustrating processing for classifying a microorganism according to Embodiment 2.

FIG. 7 is a diagram illustrating a mass spectrum of a microorganism A.

FIG. 8 is a diagram for explaining the second m/zs obtained when microorganism A is E. albertii, and when it is E. coli.

FIG. 9 is a diagram illustrating a mass spectrum of a microorganism B.

FIG. 10 is a diagram illustrating a mass spectrum of a microorganism C.

FIG. 11 is a diagram illustrating a mass spectrum of a microorganism D.

### DESCRIPTION OF EMBODIMENTS

Embodiments of the present invention will now be described in detail with reference to the accompanying drawings. It is noted that the same or corresponding components are referred to with the same reference signs in the drawings to basically avoid redundant description.

### 1. CONFIGURATION OF ANALYZER

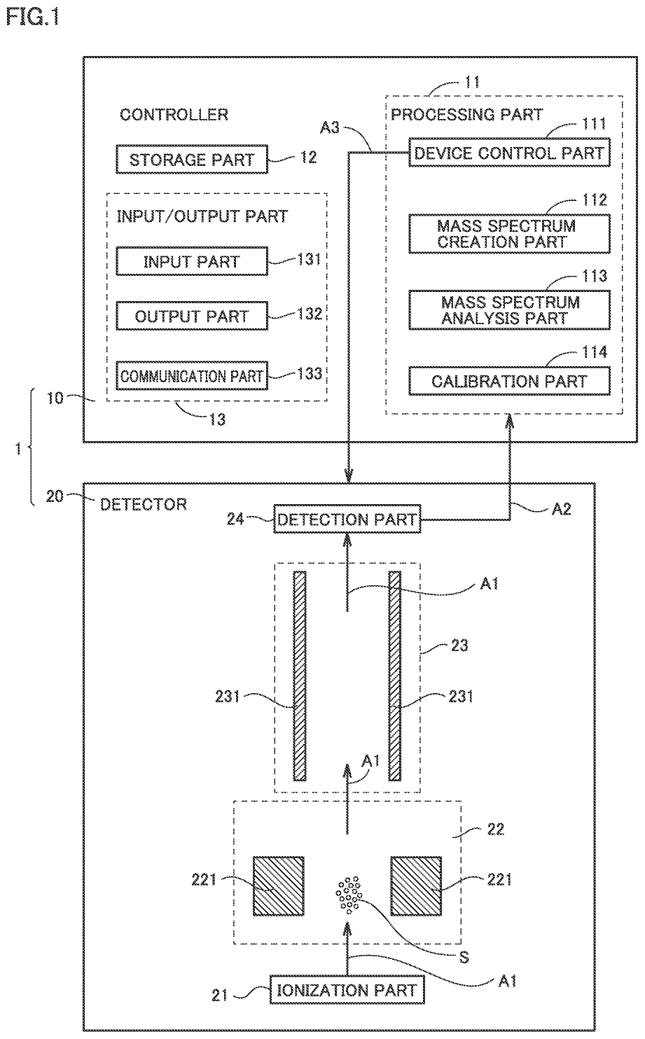

FIG. 1 is a schematic diagram illustrating the configuration of an analyzer 1 according to an embodiment of the present invention. Analyzer 1 is a mass spectrometer for performing mass spectrometry of a substance contained in a sample, and is, for example, MALDI-TOF MS (Matrix-Assisted Laser Desorption/Ionization Time-of-Flight Mass Spectrometer).

In the present embodiment, the sample is a sample derived from a microorganism belonging to the order Enterobacterales. The sample contains a target substance that is a molecule to be analyzed. The sample may contain a standard substance (calibrant) that is a molecule used for calibrating a mass spectrum. In the present embodiment, the analysis with analyzer 1 includes detecting a peak of a mass spectrum, and measuring a m/z of a specific or nonspecific substance contained in the sample. In one example, the analysis with analyzer 1 in which the substance is a protein, and the target substance is an acid shock protein may include: discriminating, based on a m/z corresponding to a peak of the mass spectrum (hereinafter, also referred to as the “actual m/z”), whether or not the specific substance is contained in the sample, calculating the concentration of the specific substance in the sample, and classifying a microorganism contained in the sample. The classifying a microorganism includes discriminating a classification category of the microorganism. Herein, classification of a microorganism refers to classification at at least one of the family, genus, species, strain levels and the like unless otherwise stated. The discriminating a classification category of the microorganism is also referred to as “identifying a microorganism”. In general, the term “identification of a microorganism” may mean identification of a microorganism at a level of the strain in a narrow sense. Herein, however, the term means a broad sense of “discriminating at least one level in the classification of a microorganism” unless otherwise stated. Moreover, herein, the classifying a microorganism includes discriminating whether or not the microorganism belongs to a prescribed classification category. The classifying a microorganism also includes discriminating whether or not a given microorganism belongs to a classification category different from that of another microorganism.

Referring to FIG. 1, analyzer 1 includes a controller 10 and a detector 20.

Detector 20 ionizes, with a high voltage, a substance (protein) contained in a sample, and detects the resultant ion S, after separation, in accordance with time of flight correlated with a m/z. Detector 20 includes an ionization part 21, an ion acceleration part 22, a mass separation part 23, and a detection part 24. In FIG. 1, the movement of the ion S in detector 20 is schematically illustrated with an arrow A1.

Ionization part 21 ionizes the substance contained in the sample by matrix-assisted laser desorption/ionization (MALDI) method. As the ionization method, not only MALDI method but also any soft ionization method such as electrospray ionization (ESI) method can be employed. In the ionization performed by ESI method, a configuration in which analyzer 1 further includes a liquid chromatograph for ionizing, with ionization part 21, a substance that is contained in the sample, and has been separated with the liquid chromatograph is preferred because high separability can be thus obtained.

Ionization part 21 includes a sample plate holder (not shown) for holding a sample plate, and an ion source including a laser device (not shown) for irradiating the sample plate with a laser beam. After placing a sample on the sample plate, a matrix is added to the sample, and the resultant sample is dried. Thereafter, the sample plate is set on the sample plate holder disposed in a vacuum container of ionization part 21. The type of the matrix is not especially limited, and from the viewpoint of efficiently ionizing a protein sample, sinapinic acid, α-cyano-4-hydroxycinnamic acid (CHCA), or the like is preferably used.

Ionization part 21 depressurizes the vacuum container in which the sample plate has been set, and then successively irradiates each sample on the sample plate with a laser beam for ionization. The type of the laser device for emitting the laser beam is not especially limited as long as it can oscillate light absorbed by the selected matrix, and for example, when the matrix contains sinapinic acid or CHCA, N2 laser (wavelength of 337 nm) or the like can be suitably used. The ion S having been ionized by ionization part 21 is extracted from an electric field formed by an extraction electrode or the like not shown, and is introduced into ion acceleration part 22.

Ion acceleration part 22 includes an accelerating electrode 221, and accelerates the ion S having been introduced thereinto. The flow of the accelerated ion S is appropriately converged by an ion lens, which are not shown, to be introduced into mass separation part 23.

Mass separation part 23 includes a flight tube 231, and separates ions S in accordance with a difference in time of flight spent by the respective ions S flying inside flight tube 231. Although FIG. 1 illustrates linear flight tube 231, a reflectron flight tube, a multi-turn flight tube or the like may be used. The method of mass spectrometry is not especially limited as long as ions S contained in a sample can be separated and detected.

Detection part 24 includes an ion detector such as a multi-channel plate, detects the ion S separated by mass separation part 23, and outputs a detected signal with an intensity according to the number of ions having entered detection part 24. The detected signal output from detection part 24 is input to a processing part 11 of controller 10. In FIG. 1, a flow of the detected signal of the ions S from detection part 24 of detector 20 is schematically illustrated with an arrow A2.

Controller 10 includes processing part 11, a storage part 12, and an input/output part 13. Controller 10 corresponds to one example of a “controller” according to the present disclosure.

Processing part 11 is configured by including a processor such as a CPU, and functions as a main part in an operation for controlling analyzer 1. Processing part 11 performs various processing by executing a program stored in storage part 12 and the like. Processing part 11 corresponds to an example of a “processor” according to the present disclosure.

Processing part 11 includes a device control part 111, a mass spectrum creation part 112, a mass spectrum analysis part 113, and a calibration part 114.

Device control part 111 controls the operation of detector 20 based on data related to analysis conditions input from an input part 131 described below. In FIG. 1, the control of detector 20 by device control part 111 is schematically illustrated with an arrow A3.

Mass spectrum creation part 112 converts the time of flight into a m/z based on measurement data including the amount of ions detected by detection part 24, and the time of flight of the ions, and creates a mass spectrum indicating a detection amount corresponding to each m/z.

Mass spectrum analysis part 113 detects, in the mass spectrum, a peak of the mass spectrum. It calculates a m/z corresponding to the detected peak. Mass spectrum analysis part 113 may discriminate, based on protein database or the like, a substance corresponding to an actual m/z indicated by the peak of the mass spectrum. In other words, mass spectrum analysis part 113 can calculate an actual m/z of a specific or nonspecific substance contained in the sample. Mass spectrum analysis part 113 may further discriminate, based on the actual m/z, whether or not the specific substance is contained in the sample (component identification in the sample), calculate the concentration of the specific substance in the sample, or classify an organism contained in the sample. More generally, mass spectrum analysis part 113 may perform structural analysis of a substance contained in the sample.

Calibration part 114 calibrates the mass spectrum based on an actual m/z and a theoretical m/z of a standard substance. The theoretical m/z is a value also referred to as a theoretical value or a theoretical m/z in general, and is a theoretical mass-to-charge ratio calculated in consideration of the molecular weight, and the number of ions and charges added. The calibration in the mass spectrometry means that the actual m/z of the standard substance is corrected to be close to the theoretical m/z, and the resultant correction is applied to the entire spectrum.

Storage part 12 includes a nonvolatile storage medium. Storage part 12 stores the theoretical m/z, the mass spectrum created by mass spectrum creation part 112, the measurement data output from detector 20, the program used for executing processing by processing part 11, and the like. Storage part 12 corresponds to an example of a “memory” according to the present disclosure.

Input/output part 13 is an interface for inputting/outputting information between analyzer 1 and the outside. Input/output part 13 includes an input part 131, an output part 132, and a communication part 133.

Input part 131 is configured by including an input device such as a mouse, a keyboard, various buttons and/or a touch panel. Input part 131 receives, from a user, information necessary for control of the operation of detector 20, and information necessary for processing performed by processing part 11.

Output part 132 is configured by including a display device such as a liquid crystal monitor, a printer, and the like. Output part 132 displays, in a display device, information on the measurement by detector 20, and results of the processing by processing part 11, or prints these on a print media.

Communication part 133 is configured by including a communication device capable of communication through wireless or wired connection such as Internet. Communication part 133 receives data necessary for processing by processing part 11, transmits data having been processed by processing part 11, such as discrimination results, and appropriately receives/transmits necessary data.

A part or the whole of the function of controller 10 described above may be disposed in a computer, a server, or the like physically separated from detector 20.

### 2. CONVENTIONAL CLASSIFICATION METHOD

Microorganisms have properties according to their classification. Microorganisms may cause environmental change in accordance with their properties, and in particular, in a body of an animal including a human, can be pathogens of various diseases. For example, it is known that a microorganism belonging to the order Enterobacterales has properties of not forming a spore in a gram-negative bacillus but producing an acid by fermenting glucose. Moreover, microorganisms belonging to the order Enterobacterales include those having pathogenicity, such as enterohemorrhagic Escherichia coli, Salmonella spp., Shigella, and Yersinia pestis, and therefore, can be a target to be considered in prevention and treatment of infectious diseases. It is also known that microorganisms belonging to the order Enterobacterales can further be a target of an epidemiological study at the occurrence of food poisoning. In other words, microorganisms belonging to the order Enterobacterales include a bacterial group important in the human society including diseases and public health.

When an infectious disease or food poisoning actually occurs, identification among the order Enterobacterales more specifically at the genus or species level, or in some cases, discrimination at the strain level in addition to the genus and species level is required. Therapeutic strategy is determined in accordance with the identified genus and species (bacterial name), or an infection route is specified based on the discrimination at the strain level, and therefore, high accuracy is required in the identification and discrimination.

For the classification of a microorganism including the identification and discrimination of a microorganism, a method using at least one of morphological characteristics and biochemical properties of the microorganism has been conventionally employed. In recent years, a method using MALDI, that is, one of mass spectrometric methods, is employed, and more accurate and faster methods have been continuously studied and developed.

In some bacterial groups, however, accurate identification is still difficult, or misidentification occurs in some cases. Moreover, discrimination at the strain level is complicated and requires a long time in many methods, and hence, there is a demand for a simpler method.

For example, some of E. coli belonging to the order Enterobacterales have a gene producing verotoxin, and cause enterohemorrhagic Escherichia coli infection. Enterohemorrhagic Escherichia coli infection may cause serious symptoms such as hemolytic uremic syndrome in some cases, and it is extremely important to rapidly and precisely specify a causative bacterium also for infection control. On the other hand, NPL 1 has reported that some of E. albertii that is a related species of E. coli also have a verotoxin gene. It has been, however, difficult to distinguish E. coli from E. albertii based on their biochemical characteristics. Intimin gene and the like are common between E. coli and E. albertii, and these bacteria may be misidentified even when a genetic test method is employed.

Moreover, even when the genus and species of a microorganism are correctly identified, more detailed analysis may be necessary in specification or the like of an infection route of food poisoning in some cases, and molecular epidemiology analysis and analysis of the difference at the strain level, namely, discrimination of the strain, is conducted. Conventional pulse field gel electrophoresis (PFGE) analysis and multi-locus sequence typing (MLST) analysis have, however, the problem of requiring a complicated operation and a long time.

### 3. ANALYSIS OF ACID SHOCK PROTEIN

As a candidate to be used as a novel index for classifying a microorganism belonging to the order Enterobacterales, the present inventors have focused on and analyzed an acid shock protein.

An acid shock protein (hereinafter also referred to as the “Asr”) refers to a specific protein known to express a gene when some microorganisms are put in acidic environments, or a protein having a sequence similar to the specific protein. As described below, the present inventors have found that the Asr includes proteins variously designated as an acid shock protein, an acid-shock protein, an acid shock repeat family protein, an acid shock-inducible periplasmic protein, an acid resistance repetitive basic protein, and a putative acid shock protein. Some of these may be hypothetical proteins having no names in some cases.

The present inventors have found, based on published gene information database and the like, that microorganisms belonging to the order Enterobacterales have a gene estimated as an Asr gene in common. The present inventors have found that the gene estimated as an Asr gene includes a gene of the protein variously designated as an acid shock protein, an acid-shock protein, an acid shock repeat family protein, an acid shock-inducible periplasmic protein, an acid resistance repetitive basic protein, or a putative acid shock protein. Based on this finding, the present inventors have made a hypothesis that the microorganisms belonging to the order Enterobacterales commonly produce the Asr. Moreover, the present inventors have presumed, based on the gene, that the Asr is diverse in the microorganisms belonging to the order Enterobacterales to an extent that classification of the genus and the species can be conducted, and in some cases, to an extent that different strains of the same genus and the same species can be classified.

In order to verify the truthfulness of the hypothesis and the presumption, the present inventors have actually cultured microorganisms of the various genus, species, and strains belonging to the order Enterobacterales, and measured and analyzed their mass spectra. Table 1 is a table of microorganisms belonging to the order Enterobacterales that the present inventors have used for the analysis.

TABLE 1

Family Enterobacteriaceae
Escherichia coli
Escherichia albertii
Shigella dysenteriae
Salmonella enterica
Cronobacter sakazakii
Enterobacter asburiae
Family Erwiniaceae
Erwinia aphidicola
Erwinia persicina
Pantoea agglomerans
Family Pectobacteriaceae
Pectobacterium carotovorum
Family Yersinia
Yersinia bercovieri
Yersinia rohdei
Yersinia ruckeri
Serratia ficaria
Rahnella aquatilis
Family Hafnia
Hafnia alvei
Edwardsiella tarda
Family Morganellaceae
Morganella morganii
Proteus hauseri
Providencia alcalifaciens
Cosenzaea myxofaciens
Family Budubisiaceae
Budvicia aquatica
Leminorella grimontii
Pragia fontium

As a result of the analysis, the present inventors have found that the microorganisms belonging to the order Enterobacterales produce the Asr without exception when grown under prescribed conditions. The present inventors have further found that an amino acid sequence presumed from the Asr gene is cleaved at a specific amino acid sequence (Gln-Lys-Ala-Gln sequence) to be fragmented, and that the resultant fragment can be easily and clearly observed by mass spectrometry. The present inventors have further found that the Asrs produced respectively by microorganisms belonging to different classification categories of the order Enterobacterales have different actual m/zs. In other words, the present inventors have found that microorganisms belonging to the order Enterobacterales have characteristics having diversity in the Asr according to their classification categories, and that the diversity is reflected in their mass spectra.

The present inventors have constructed, based on the characteristics, a method for classifying a microorganism of the present embodiment.

### 4. METHOD FOR CLASSIFYING MICROORGANISM OF EMBODIMENT

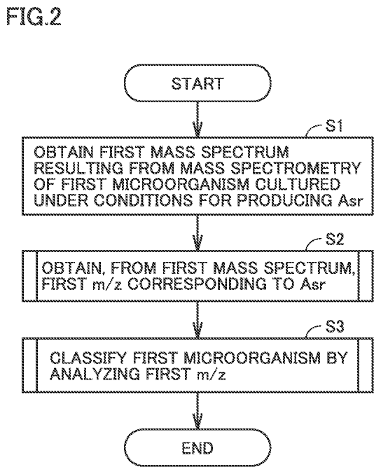

FIG. 2 is a flowchart illustrating processing for classifying a microorganism of the present embodiment. Respective steps illustrated in FIG. 2 are executed by analyzer 1. It is noted that “S” used in the drawing is used as an abbreviation of “STEP”.

In S1, analyzer 1 obtains a mass spectrum resulting from mass spectrometry of a microorganism belonging to the order Enterobacterales that has been cultured under conditions for producing an Asr, and classification of the microorganism at and below one of the family, genus, and species levels is unknown. The “microorganism belonging to the order Enterobacterales, and classification of the microorganism at and below one of the family, genus, and species levels is unknown” will be hereinafter also referred to as the “first microorganism”. In other words, the first microorganism is any one of a microorganism whose classification at and below the family level is unknown, a microorganism whose classification at and below the genus level is unknown, and a microorganism whose classification at and below the species level is unknown. Moreover, the “mass spectrum resulting from mass spectrometry of the first microorganism” will be hereinafter also referred to as the “first mass spectrum”.

The conditions for producing an Asr include at least one of a condition that involves culturing in a medium supplemented with a sugar, a condition that involves employing an anaerobic state, and a condition that involves performing prolonged culture. The term “microorganism whose classification is unknown” means that the classification is unknown to a user of a method for classifying the microorganism of the present embodiment, and does not always refer to an unknown microorganism that has never been identified in classification.

The condition that involves culturing in a medium supplemented with a sugar can be satisfied, for example, by adding a sugar in creating a medium to be used for the culture. The sugar to be added to the medium is not especially limited, and is preferably a monosaccharide, a disaccharide, a trisaccharide, and a tetrasaccharide. A sugar obtained by binding 5 or more monosaccharides may be used. The monosaccharide to be added to the medium is at least one of arose, altrose, glucose, mannose, gulose, idose, galactose, talose, fucose, fuculose, rhamnose, psicose (also referred to as allulose), fructose, sorbose, tagatose, ribose, arabinose, xylose, lyxose, ribulose, xylulose, deoxyribose, sedoheptulose, ketotetrose, erythrulose, aldotetrose, erythrose, threose, ketotriose (dihydroxyacetone), and aldotriose (glyceraldehyde). Ketotetrose is preferably erythrulose. Aldotetrose is preferably erythrose or threose. One sugar out of these monosaccharides may be added, or a combination of a plurality of these sugars may be used. The sugar is more preferably glucose. The disaccharide to be added to the medium is, for example, at least one of sucrose, lactose, maltose, trehalose, turanose, and cellobiose, and is preferably lactose. The trisaccharide to be added to the medium is, for example, at least one of raffinose, melezitose, and maltotriose. The tetrasaccharide to be added to the medium is, for example, at least one of acarbose and stachyose. The range of the concentration of the sugar to be added to the medium is preferably 0.1 wt % or more, and more preferably 0.5 wt % or more in the medium.

The condition that involves employing an anaerobic state is satisfied by, for example, culture in an oxygen concentration of 5% or less (preferably 1% or less).

Herein, the prolonged culture refers to culture performed for a prescribed time period or longer, which is longer than a usual time period for culturing a microorganism to be used in an experiment. The usual culture period and the specific time period of the prolonged culture can be, however, different depending on microorganisms. Therefore, the condition that involves performing prolonged culture can be different depending on microorganisms, and can be satisfied, for example, by culturing a microorganism, which is usually cultured overnight before use in an experiment, over two nights. More specifically, the condition is satisfied by culturing a microorganism, which is usually cultured for about 12 to 18 hours before use in an experiment, for about 36 to 42 hours.

In S1, the first mass spectrum is obtained, for example, by measuring a sample containing the first microorganism with detector 20. The method for obtaining the first mass spectrum is not limited to this, and for example, it may be obtained from the outside of analyzer 1 through input part 131 or communication part 133.

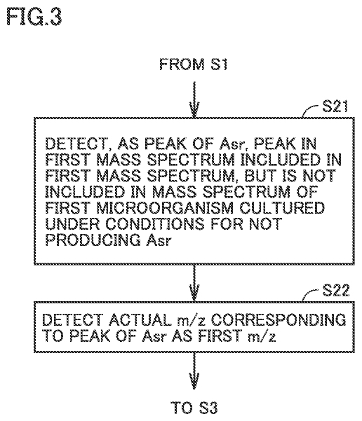

In S2, analyzer 1 obtains a first m/z, that is, an actual m/z corresponding to the Asr, from the first mass spectrum. Detailed steps for obtaining the first m/z will be described below referring to FIG. 3.

In S3, analyzer 1 analyzes the first m/z to classify the first microorganism at or below one of the unknown levels.

FIG. 3 is a flowchart illustrating processing for obtaining the first m/z. S21 to S22 illustrated in FIG. 3 are processing corresponding to S2 of FIG. 2. The respective steps illustrated in FIG. 3 are executed in analyzer 1. Through the respective steps illustrated in FIG. 3, a user can detect a peak of the Asr by a simple method.

In S21, analyzer 1 detects, as a peak of the Asr, a peak in the first mass spectrum wherein the peak is included in the first mass spectrum, but is not included in a mass spectrum of the first microorganism cultured under conditions for not producing the Asr. The conditions for not producing the Asr refer to conditions that do not satisfy the conditions for producing the Asr. In other words, the conditions for not producing the Asr are conditions at least satisfying none of the condition that involves culturing in a medium supplemented with a sugar, the condition that involves employing an anaerobic state, and the condition that involves performing prolonged culture.

In S22, analyzer 1 detects a m/z corresponding to the peak of the Asr as the first m/z.

According to the method for classifying a microorganism illustrated in FIG. 2, the first microorganism can be classified at the family, genus, or species level by analyzing the first m/z corresponding to the Asr of the first microorganism. In other words, a microorganism belonging to the order Enterobacterales can be classified by a simple method.

As described so far, in the method for classifying a microorganism according to the present embodiment, any microorganism belonging to the order Enterobacterales can be classified based on a m/z pattern of an Asr thereof. Subsequently, a method for classifying an unknown microorganism based on a theoretical m/z of an Asr, and a method for discriminating a plurality of microorganisms based on actual m/zs of Asrs encompassed in the present embodiment will be successively described.

### 4-1. Embodiment 1

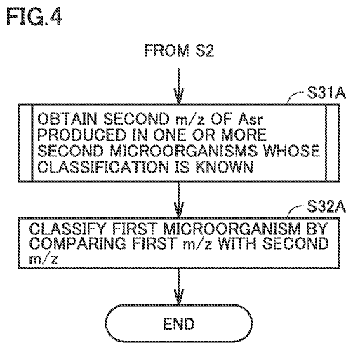

FIG. 4 is a flowchart illustrating processing for classifying a microorganism according to Embodiment 1. S31A to S32A illustrated in FIG. 4 are processing corresponding to S3 of FIG. 2. The respective steps illustrated in FIG. 4 are executed in analyzer 1.

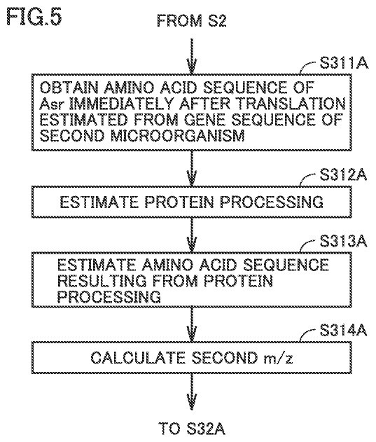

In S31A, analyzer 1 obtains a theoretical m/z of an Asr produced in one or more microorganisms belonging to the order Enterobacterales, classification of the microorganisms at the family, genus, and species levels being known. The “microorganism belonging to the order Enterobacterales, classification of the microorganism at the family, genus, and species levels being known” will be hereinafter also referred to as the “second microorganism”. Moreover, the “theoretical m/z of an Asr produced in the second microorganism” will be hereinafter also referred to as the “second m/z”. Detailed steps for obtaining the second m/z will be described below referring to FIG. 5.

In S32A, analyzer 1 compares the first m/z with the second m/z to classify the first microorganism.

In one example, the second microorganism is a microorganism estimated by a user as belonging to the same classification category as the first microorganism. For example, a user estimates the classification category at an unknown level of the first microorganism. This estimation of the classification is conducted, for example, through observation of the form of the first microorganism, by a property test, and based on growing environmental conditions. Then, one or more microorganisms out of microorganisms belonging to the estimated classification category serve as the second microorganism.

As a more specific example, when the classification at the family to strain levels of the first microorganism is estimated by a user, one or more microorganisms out of microorganisms belonging to the estimated classification category at the family to strain levels serve as the second microorganism. In this specific example, when the first m/z corresponds to the second m/z, namely, when the actual m/z of the first microorganism matches the theoretical m/z of the second microorganism estimated as the identity of the first microorganism, analyzer 1 determines that the first microorganism and the second microorganism belong to the same classification category at all the family, genus, species, and strain levels. In other words, it is determined that the first microorganism is a microorganism belonging to the estimated family, genus, species, and strain. In this case, it is deemed that the first microorganism is identified at the strain level. In other words, it is deemed that the second microorganism is the identity of the first microorganism at the strain level.

On the other hand, in this specific example, when the first m/z does not correspond to the second m/z, namely, when the actual m/z of the first microorganism does not match the theoretical m/z of the second microorganism estimated as the identity of the first microorganism, analyzer 1 determines that the first microorganism and the second microorganism are of different classification categories at and below one of the family, genus, species, and strain levels. In other words, it is determined that the first microorganism is not a microorganism belonging to at least the estimated strain. In other words, it is at least understood that the first microorganism is a microorganism belonging to a strain other than the estimated strain. In this case, the user estimates a classification category of the first microorganism different from that previously estimated, and repeatedly compares the first m/z with a second m/z of a new second microorganism belonging to the different classification category again, and thus, the identification can be conducted.

A plurality of microorganisms of the order Enterobacterales that are respectively of different classification categories at and below one of the family, genus, and species levels may be estimated as the second microorganisms from the beginning. In this case, in S32A, analyzer 1 classifies the first microorganism based on the first m/z and the theoretical m/zs of Asrs of the plurality of different microorganisms. In this case, analyzer 1 may compare, similarly to the above-described example, the theoretical m/z and the first m/z with respect to each of the plurality of microorganisms. On the other hand, analyzer 1 may compare the theoretical m/zs of the plurality of microorganisms with the first m/z at a time. For example, a microorganism having a theoretical m/z closest to the first m/z among the theoretical m/zs of the Asrs of the plurality of microorganisms is extracted, and the theoretical m/z of the extracted microorganism may be compared with the first m/z. In such configuration, the first m/z can be compared at a time with the plurality of second m/zs respectively corresponding to the plurality of second microorganisms respectively belonging to different classification categories, and therefore, the first microorganism can be more efficiently classified as compared with a case where the first m/z is compared with the second m/z corresponding to one of the second microorganisms.

As another specific example, when the classification of the first microorganism at the family to species levels are estimated by a user, one or more microorganisms out of microorganisms belonging to the estimated classification category at the family to species levels serve as the second microorganism. For example, in S32A, when a similar theoretical m/z pattern of the Asr is found in a plurality of second microorganisms belonging to the estimated species, it can be found, depending on whether or not the pattern is found also in the first m/z, whether or not the first microorganism belongs to the estimated species. For example, when the pattern is found in the first m/z, it is suggested that the first microorganism may belong to the estimated species. When the family of the first microorganism is estimated by a user, again, it can be similarly found whether or not the first microorganism belongs to the estimated family.

In another example, the second microorganism may be chosen without the estimation of the classification by a user. For example, when a user has no knowledge about the classification of the first microorganism, the user cannot estimate the classification of the first microorganism. In this case, again, the first microorganism can be classified by comparing the first microorganism with the second microorganism. However, excluding a case where the number of the second microorganisms is sufficiently large, and a case where the speed of the processing for comparing the first microorganism and the second microorganism in analyzer 1 is high, it is more efficient, for identifying the microorganism, to choose the second microorganism based on the estimation of the classification of the first microorganism.

FIG. 5 is a flowchart illustrating processing for obtaining a second m/z. S311A to S314A illustrated in FIG. 5 are processing corresponding to S31A of FIG. 4. The respective steps illustrated in FIG. 5 are executed in analyzer 1.

In S311A, analyzer 1 obtains an amino acid sequence of the Asr immediately after translation estimated from the gene sequence of the second microorganism. For example, a gene having a characteristic of the Asr is estimated by analyzer 1, in published gene database, as the gene of the Asr to obtain the gene sequence of the Asr, and the amino acid sequence of the Asr immediately after translation is estimated based on it. When analyzer 1 can obtain the amino acid sequence itself of the second microorganism immediately after the translation estimated from the gene sequence, the amino acid sequence immediately after the translation itself may be obtained.

In S312A, analyzer 1 estimates protein processing to be performed on the amino acid sequence immediately after translation. The protein processing includes removal of a signal peptide, and cleavage on the C-terminal side of Gln-Lys-Ala-Gln sequence. This step is based on the characteristic, found by the present inventors, that an Asr of a microorganism belonging to the order Enterobacterales is cleaved on the C-terminal side of Gln-Lys-Ala-Gln sequence.

In S313A, analyzer 1 estimates an amino acid sequence resulting from the protein processing based on the amino acid sequence immediately after translation, and the estimated protein processing.

In S314A, analyzer 1 calculates a second m/z based on the amino acid sequence resulting from the protein processing.

As illustrated in FIG. 3, when the protein processing including the removal of a signal peptide, and the cleavage on the C-terminal side of Gln-Lys-Ala-Gln sequence is considered, the second m/z can be more accurately calculated than when it is not considered.

According to the method for classifying a microorganism of Embodiment 1, a first microorganism can be classified by comparing an actual m/z of the first microorganism whose classification at and below one of the family, genus, and species levels is unknown with a theoretical m/z of one or more second microorganisms whose classification at the family, genus, and species levels is known. Moreover, in one aspect of the classification, the first microorganism can be identified.

### 4-2. Embodiment 2

Subsequently, as another embodiment of the method for classifying a first microorganism, a method for distinguishing a plurality of microorganisms in accordance with patterns of Asrs in mass spectra will be described.

In medical and research settings, it is, in some cases, necessary to distinguish a plurality of microorganisms from one another. An example of the plurality of microorganisms includes one microorganism whose classification at and below one of the family, genus, species, and strain levels is unknown to a user (corresponding to the first microorganism), and one microorganism whose classification at or below one of the family, genus, and species levels is known. Another example of the plurality of microorganisms includes two microorganisms whose classification at all the family, genus, and species levels are unknown to a user. A method for distinguishing these plurality of microorganisms based on the diversity of an Asr will now be described.

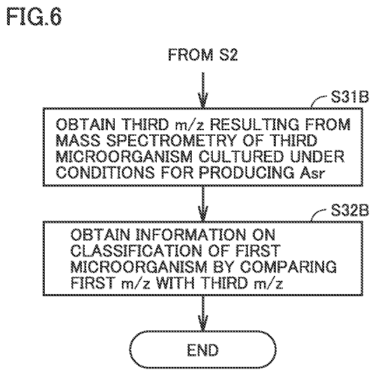

FIG. 6 is a flowchart illustrating processing for classifying a microorganism according to Embodiment 2. S31B to S32B illustrated in FIG. 6 are processing corresponding to S3 of FIG. 2. The respective steps illustrated in FIG. 6 are executed in analyzer 1.

In S31n, analyzer 1 obtains an actual m/z of an Asr produced in a microorganism, obtained by mass spectrometry of the microorganism that have been cultured under conditions for producing an Asr wherein the microorganism is one or more microorganisms belonging to the order Enterobacterales. The “one or more microorganisms belonging to the order Enterobacterales” will be hereinafter referred to also as the “third microorganism”. The “actual m/z of an Asr produced in the third microorganism” will be hereinafter referred to also as the “third m/z”. A detailed step for obtaining the third m/z corresponds to the step for obtaining the first m/z illustrated in FIG. 3.

In S32B, analyzer 1 obtains information on the classification of the first microorganism by comparing the first m/z with the third m/z.

In one example, the third microorganism is a microorganism whose classification at or below one of the family, genus, and species levels is known. Now, some of examples of situations in which it is necessary to distinguish the first microorganism from the third microorganism whose classification is known will be described. A first example is a situation in which it is desired to determine whether a microorganism whose classification is unknown (first microorganism) present in an environment where a microorganism known to belong to at least one of prescribed family, genus, and species (third microorganism) is considered to be present is the third microorganism, or another microorganism. A second example is a situation in which the classification of a microorganism whose classification is unknown (first microorganism) is estimated, a mass spectrum of a microorganism known to belong to the classification category (third microorganism) can be obtained, and it is desired to confirm whether the identity of the first microorganism is the third microorganism. A third example is a situation in which it is desired to examine a possibility that a microorganism whose classification is unknown (first microorganism) and undistinguishable, based on the appearance and a property test, from a microorganism whose classification is known (third microorganism) is actually a microorganism different from the third microorganism. A fourth example is a case in which it is desired to obtain any knowledge about the classification of a microorganism whose classification is unknown (first microorganism) by comparing the first microorganism with a microorganism whose classification at a nonspecific level is known (third microorganism). The situation in which it is necessary to distinguish a first microorganism whose classification is unknown from a third microorganism whose classification is known is not limited to these examples, and may be any situation in which a person skilled in the art who classifies microorganisms needs the discrimination.

As a more specific example, a case in which the classification of the third microorganism at the family to strain levels is known will be described. The present inventors have analyzed genes of microorganisms of the order Enterobacterales obtained from published gene information database, and have found that even when the genus and the species are the same, amino acid sequences estimated from genes estimated as Asr genes are different among strains in many cases. Moreover, the present inventors have verified, through measurement and analysis of mass spectra, the difference in the amino acid sequence as a difference in the molecular weight. Based on this characteristic, when the first m/z and the third m/z are different from each other, it can be determined in step S32B that the first microorganism and the third microorganism are of different strains. In other words, it can be confirmed that the first microorganism is a microorganism belonging to a strain different from that of the third microorganism. When the first m/z and the third m/z are the same, it cannot be determined that these are of the same strain, but the possibility is suggested. In consideration of this possibility, the user can attempt to determine whether or not these are of the same strain by another method. When the first m/z and the third m/z are different, again, the first microorganism can be identified by repeatedly comparing the first m/z with a third m/z of a new third microorganism belonging to a classification category different from that of the previous case again.

It is noted that the third microorganism may be a plurality of microorganisms of the order Enterobacterales different at and below one of the family, genus, and species levels. In this case, in S32B, analyzer 1 obtains information on the classification of the first microorganism based on the first m/z and m/zs of Asrs of mass spectra of the plurality of different microorganisms. In this case, analyzer 1 may compare the actual m/z of each of the plurality of microorganisms with the first m/z, or may compare the actual m/zs of the plurality of microorganisms with the first m/z at a time. In such configuration, the first m/z can be compared at a time with the plurality of actual m/zs respectively corresponding to the plurality of third microorganisms respectively belonging to different classification categories, and therefore, the first microorganism can be more efficiently classified as compared with a case where the first m/z is compared with the third m/z corresponding to one of the third microorganisms.

As described so far, in the method for classifying a microorganism illustrated in FIG. 6, discrimination between strains is possible which determines whether microorganisms of the same genus and the same species are of the same strain by comparing the microorganisms in detail.

As a different specific example, the third microorganism may be a microorganism whose classification at the family to species levels is known. For example, in S32B, when a pattern of the actual m/z of a similar Asr is found in a plurality of second microorganisms belonging to the estimated species, it can be known, depending on whether or not the pattern is found in the first m/z, whether or not the first microorganism belongs to the estimated species. For example, when the pattern is found in the first m/z, a possibility that the first microorganism belongs to the species to which the third microorganism belongs is suggested. When the family of the third microorganism is known, again, it can be similarly known whether or not the first microorganism belongs to the family to which the third microorganism belongs.

In another example, the third microorganism may be a microorganism whose classification at all the family, genus, species, and strain levels is unknown. It is useful to determine whether or not microorganisms are distinguishable even if their classification is unknown. For example, in S32B, when the first m/z and the third m/z correspond to each other, and it is presumed that there is a possibility that the microorganisms are of the same strain, it is presumed that there is a possibility that these can be similarly dealt with. As a more specific example, when microorganisms collected from different environments are actually identical, it is presumed that there is a possibility that a drug against the same microorganism may be effective. In S32B, however, when the first m/z and the third m/z do not correspond to each other, and the microorganisms are of different strains, it is presumed that there is a possibility that these need to be dealt with differently. Moreover, when a group of unknown microorganisms is found, it is important to determine whether or not microorganisms included in the group of microorganisms are mutually distinguishable for confirming what classification category is present therein.

According to the method for classifying a microorganism of Embodiment 2, information on the classification of a plurality of microorganisms can be obtained by comparing patterns of peaks of Asrs in mass spectra of a plurality of microorganisms (a first microorganism and a third microorganism) belonging to the order Enterobacterales. In particular, the plurality of microorganisms can be mutually distinguished as long as these are not identical.

### 5. EXAMPLES

### 5-1. Identification of E. albertii

Next, examples conducted for verifying the effects of the present invention will be described.

In this example, it will be described that E. albertii can be correctly identified without being misidentified as E. coli that is a related species thereof. There is a possibility that these bacteria may produce verotoxin, and these are important as a bacterium that can be a causative bacterium of food poisoning and infectious diseases. Moreover, it has been difficult to correctly distinguishably identify E. albertii and E. coli by a conventional biochemical property test.

As a strain to be used in this example, NBRC 107761 was obtained from NITE Biological Resource Center. This strain is a standard strain of E. albertii, but the actual identification is performed with the strain name unknown, and hence, this strain is referred to as a microorganism A here. Microorganism A corresponds to the “first microorganism”.

Microorganism A was cultured in sugar-supplemented medium, IFO 804 medium (0.5 wt % of hipolypepton, 0.5 wt % of yeast extract, 0.5 wt % of glucose, 0.1 wt % of magnesium sulfate) used as a medium satisfying the conditions for producing an Asr. Moreover, microorganism A was cultured also in IFO 802 medium not supplemented with sugar (1 wt % of hipolypepton, 0.2 wt % of yeast extract, 0.1 wt % of magnesium sulfate) used as a medium satisfying the conditions for not producing an Asr. From these cultured bacteria, proteins and the like were extracted with trifluoroacetic acid (TFA) to be used as samples, and mass spectra thereof were obtained with a mass spectrometer (manufactured by Shimadzu Corporation, MALDI-8020).

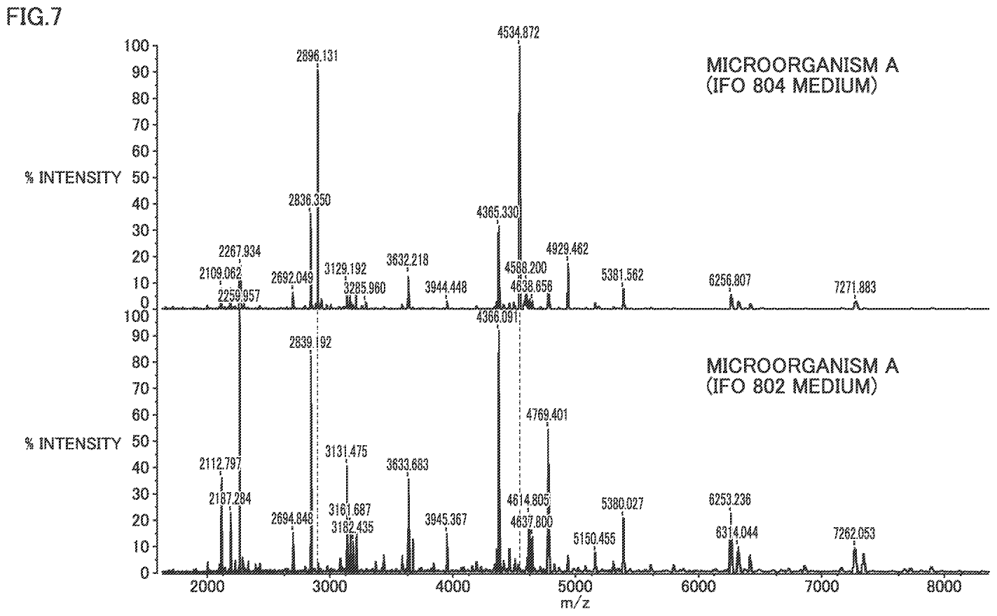

FIG. 7 is a diagram illustrating the mass spectra of microorganism A. The mass spectrum of microorganism A having been cultured in IFO 804 medium is illustrated in the upper portion of FIG. 7, and the mass spectrum of microorganism A having been cultured in IFO 802 medium is illustrated in the lower portion. Comparing these, there are peaks clearly found only in the mass spectrum of the upper portion, and the actual m/zs of the peaks are respectively 2896.1 and 4534.9. These actual m/zs correspond to the “first m/z”.

Next, amino acid sequences of Asrs of E. albertii and E. coli were obtained from NCBI. Protein_ids of E. albertii and E. coli in NCBI were respectively GAL55325.1 and AY071325.1. Here, E. albertii and E. coli correspond to the “second microorganism”.

A signal peptide was removed from the amino acid sequence of each of E. albertii and E. coli, and when Gln-Lys-Ala-Gln sequence (corresponding to QKAQ sequence in the amino acid one letter code) was present in a partial amino acid sequence of the remaining main body of the protein, the rule of cleavage on the carboxyl group terminal side (C-terminal side) thereof was applied, and thus, the sequence of a protein fragment finally produced was determined. Moreover, based on the amino acid sequence of the fragment, the molecular weight and the theoretical m/z were calculated. The theoretical m/zs thus obtained correspond to the “second m/z”.

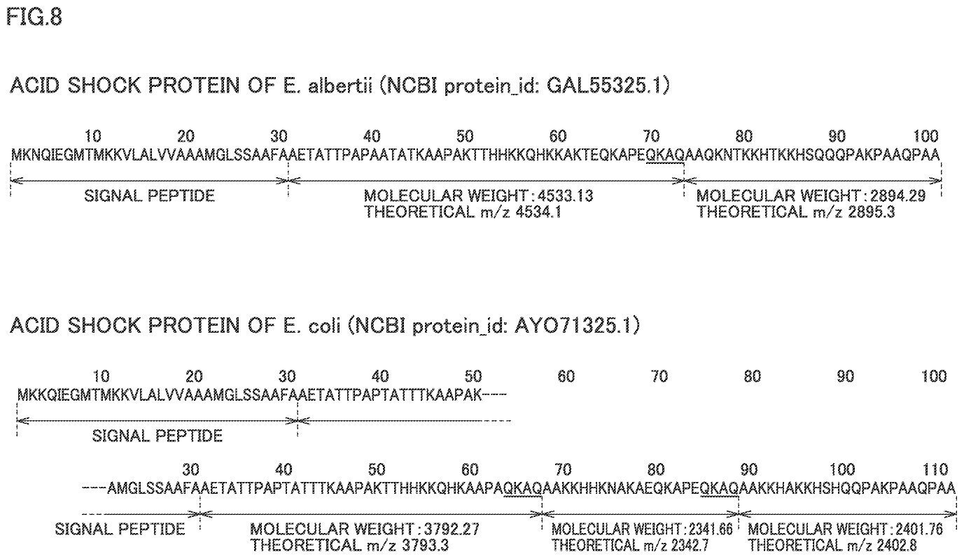

FIG. 8 is a diagram for explaining the second m/z obtained when microorganism A is E. albertii, and when it is E. coli. In FIG. 8, two mass spectral peaks derived from Asrs were observed when microorganism A was E. albertii, and the m/zs thereof were estimated as 2895.3 and 4534.1. On the other hand, when microorganism A was E. coli, three mass spectral peaks were observed, and the m/zs thereof were estimated as 2342.7, 2402.8, and 3793.3.

As described above, in the actual mass spectrum of microorganism A in FIG. 7, the number of Asr-derived peaks was two, and the actual m/zs thereof were 2896.1 and 4534.9, and thus, the number was the same as that estimated when microorganism A was E. albertii, and the first m/z was substantially the same as the second m/z estimated in that case. Accordingly, microorganism A could be correctly identified as E. albertii.

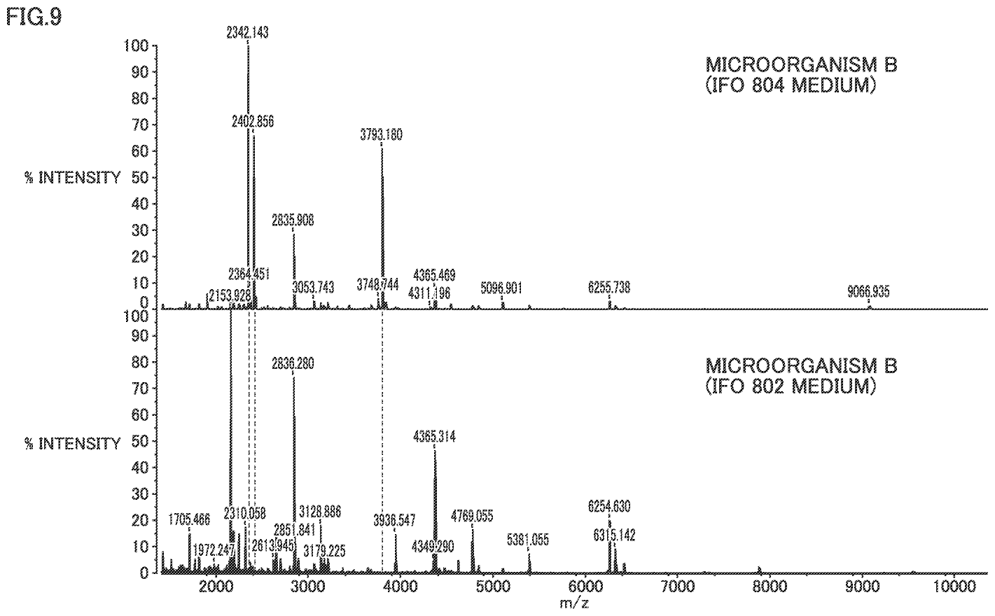

As another example, mass spectra obtained by assuming that E. coli is a microorganism B that is another first microorganism are illustrated in FIG. 9. In this case, again, mass spectral peaks in number estimated when microorganism B was E. coli were observed in microorganism B, and the first m/z was substantially the same as the second m/z estimated in that case. In this manner, a microorganism belonging to the order Enterobacterales can be identified based on the diversity of Asrs.

### 5-2. Discrimination of Hafnia Alvei

In this example, it is described that two strains of Hafnia alvei (hereinafter also referred to as H. alvei) can be distinguished from each other based on a method for identifying a microorganism of the present embodiment.

As a strain to be used in this example, NBRC 3731 and NBRC 105685 were obtained from NITE Biological Resource Center. NBRC 105685 is a standard strain of H. alvei, but NBRC 3731 is not described as a standard strain, and hence is estimated as a different strain. The actual discrimination is, however, performed in a state where the classification of the microorganisms is unknown, and hence, these microorganisms are referred to as a microorganism C and a microorganism D in this example for convenience. One of microorganisms C and D corresponds to the “first microorganism”, and the other corresponds to the “third microorganism”. The following description will be given assuming that microorganism C is the “first microorganism”, and that microorganism D is the “third microorganism”.

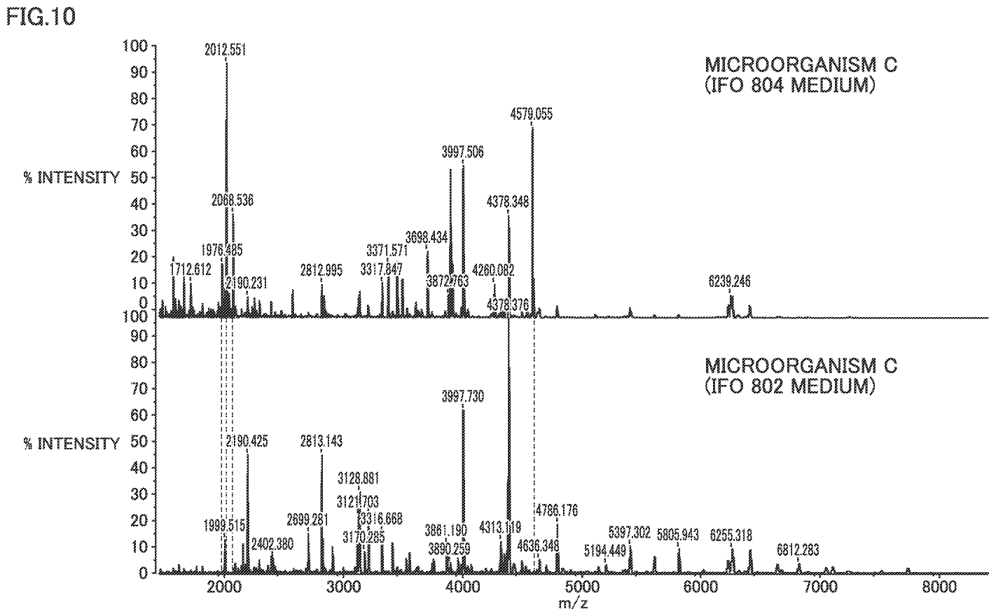

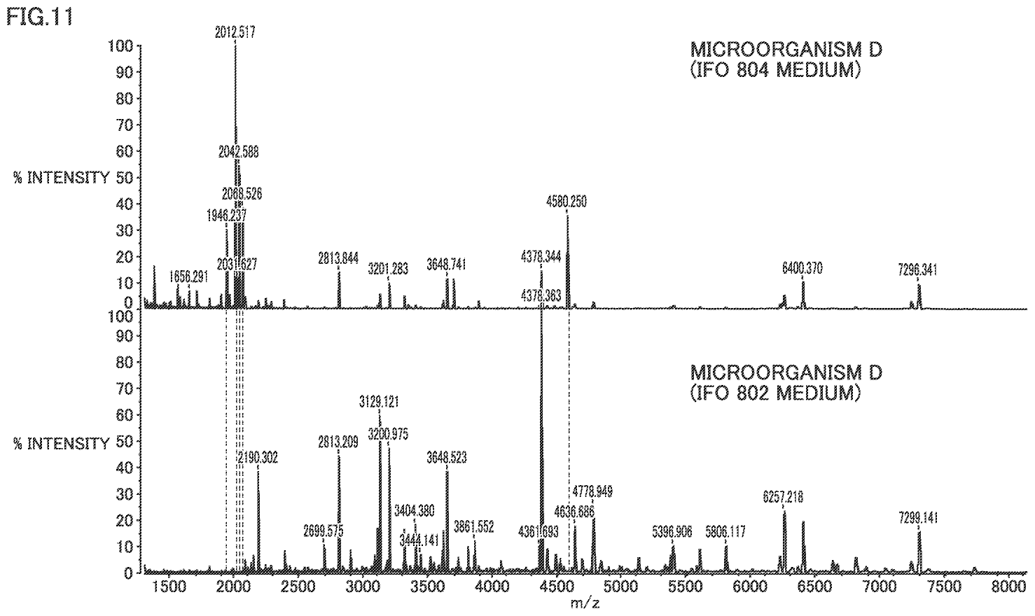

Each of microorganism C and microorganism D was cultured in a sugar-supplemented medium, IFO 804 medium (0.5 wt % of hipolypepton, 0.5 wt % of yeast extract, 0.5 wt % of glucose, 0.1 wt % of magnesium sulfate) used as a medium satisfying the conditions for producing an Asr. Moreover, each of microorganism C and microorganism D was cultured also in IFO 802 medium not supplemented with sugar (1 wt % of hipolypepton, 0.2 wt % of yeast extract, 0.1 wt % of magnesium sulfate) used as a medium satisfying the conditions for not producing an Asr. From these cultured bacteria, proteins and the like were extracted with trifluoroacetic acid (TFA) to be used as samples, and mass spectra thereof illustrated in FIG. 10 and FIG. 11 were obtained with a mass spectrometer (manufactured by Shimadzu Corporation, MALDI-8020).

FIG. 10 and FIG. 11 are diagrams respectively illustrating mass spectra of microorganism C and microorganism D. The mass spectra of microorganism C and microorganism D having been cultured in IFO 804 medium are illustrated in the upper portions of FIG. 10 and FIG. 11, and the mass spectra of microorganism C and microorganism D having been cultured in IFO 802 medium are illustrated in the lower portions. In each of FIG. 10 and FIG. 11, comparing the mass spectra in the upper portion and the lower portion, there are peaks clearly found only in the mass spectrum of the upper portion, which are estimated to be derived from an Asr fragment.

The actual m/zs (corresponding to the “first m/z”) of Asrs of microorganism C were 1976.5, 2012.6, 2068.5, and 4579.1. On the other hand, the actual m/zs (corresponding to the “third m/z”) of Asrs of microorganism D were 1946.2, 2012.5, 2042.6, 2068.5, and 4580.3. These are listed in Table 2 below.

TABLE 2

| Microorganism C | Microorganism D |
|---|---|
| | 1946.2 |
| 1976.5 | |
| 2012.6 | 2012.5 |
| | 2042.6 |
| 2068.5 | 2068.5 |
| 4579.1 | 4580.3 |

As shown in Table 2, in microorganism C and microorganism D having been produced in the medium satisfying the conditions for producing an Asr, unmatched peaks were detected. This indicates that there was a difference in a partial amino acid sequence of the Asr, and can be clear evidence that microorganism C and microorganism D belong to different classification categories. Accordingly, it can be determined that microorganism C and microorganism D are of different strains.

Although the discrimination performed when microorganism C and microorganism D were of different strains of the same species of Hafnia alvei in the example, it is obvious that the discrimination can be similarly performed also when microorganism C and microorganism D belong to different species, different genera, and different families.

### ASPECTS

Those skilled in the art will understand that the above-described plurality of exemplifying embodiments are specific examples of the following aspects.

(Item 1) A method for classifying a microorganism according to one aspect is a method for classifying a microorganism, including: obtaining a first mass spectrum resulting from mass spectrometry of a first microorganism belonging to the order Enterobacterales that has been cultured under conditions for producing an acid shock protein, classification of the first microorganism at and below one of the family, genus, and species levels being unknown; obtaining a first m/z corresponding to the acid shock protein from the first mass spectrum; and performing classification at or below one of the unknown levels of the first microorganism by analyzing the first m/z.

In the method for classifying a microorganism according to item 1, the first microorganism can be classified at or below one of the unknown levels. Therefore, a microorganism belonging to the order Enterobacterales can be classified by a simple method.

(Item 2) In the method for classifying a microorganism according to item 1, the performing classification at or below one of the unknown levels includes: obtaining a second m/z of an acid shock protein produced in one or more second microorganisms of the order Enterobacterales, classification of the second microorganisms at the family, genus, or species level being known; and performing classification of the first microorganism by comparing the first m/z with the second m/z.

In the method for classifying a microorganism according to item 2, it can be known whether or not the first microorganism belongs to the same family, genus, or species as the second microorganism.

(Item 3) In the method for classifying a microorganism according to item 2, classification categories at all of the family, genus, species, and strain levels of the second microorganisms are known, and when the first m/z corresponds to the second m/z, the performing classification of the first microorganism includes determining that the first microorganism belongs to the same classification categories at all of the family, genus, species, and strain level as any of the second microorganisms.

In the method for classifying a microorganism according to item 3, the first microorganism can be classified at the strain level. Moreover, in one aspect of the classification, the first microorganism can be identified at the strain level.

(Item 4) In the method for classifying a microorganism according to item 2 or 3, the second microorganisms are a plurality of microorganisms of the order Enterobacterales different from one another at and below one of the family, genus, and species levels, and the performing classification of the first microorganism includes performing the classification of the first microorganism based on the first m/z, and m/zs of acid shock proteins of the plurality of different microorganisms.

In the method for classifying a microorganism according to item 4, the first m/z can be compared, at a time, with the plurality of second m/zs respectively corresponding to the plurality of second microorganisms belonging to different classification categories, and hence the first microorganism can be more efficiently classified as compared with a case where the first m/z is compared with the second m/z corresponding to one of the second microorganisms.

(Item 5) In the method for classifying a microorganism according to any one of items 1 to 4, the obtaining the first m/z includes: detecting, as a peak of the acid shock protein, a peak in the first mass spectrum wherein the peak is included in the first mass spectrum, but is not included in a mass spectrum of the first microorganism cultured under conditions for not producing the acid shock protein; and detecting a m/z corresponding to the peak of the acid shock protein as the first m/z.

In the method for classifying a microorganism according to item 5, a peak of the acid shock protein can be detected by a simple method.

(Item 6) In the method for classifying a microorganism according to any one of items 2 to 5, the obtaining the second m/z includes: obtaining an amino acid sequence of the acid shock protein immediately after translation estimated from a gene sequence of the second microorganisms; estimating protein processing to be performed on the amino acid sequence immediately after translation; estimating an amino acid sequence resulting from the protein processing; and calculating the second m/z based on the amino acid sequence resulting from the protein processing, and the protein processing includes removal of a signal peptide, and cleavage on a C-terminal side of Gln-Lys-Ala-Gln sequence.

In the method for classifying a microorganism according to item 6, the second m/z can be more accurately calculated by considering the protein processing including the removal of a signal peptide, and the cleavage on the C-terminal side of Gln-Lys-Ala-Gln sequence as compared with a case where it is not considered.

(Item 7) In the method for classifying a microorganism according to item 1, the performing classification at or below one of the unknown levels includes: obtaining a third m/z of an acid shock protein produced in a third microorganism, the third m/z being obtained by mass spectrometry of the one or more third microorganisms that have been cultured under conditions for producing an acid shock protein wherein the third microorganisms belonging to the order Enterobacterales; and obtaining information on the classification of the first microorganism by comparing the first m/z with the third m/z.

In the method for classifying a microorganism according to item 7, through comparison of patterns of peaks of Asrs in mass spectra of a plurality of microorganisms (the first microorganism and third microorganism) belonging to the order Enterobacterales, information on the classification of the plurality of microorganisms can be obtained. In particular, the microorganisms can be thus distinguished from each other as long as the plurality of microorganisms are not identical.

(Item 8) In the method for classifying a microorganism according to item 7, the obtaining the information includes determining that the first microorganism and the third microorganism are of different strains when the first m/z is different from the third m/z.

In the method for classifying a microorganism according to item 8, it can be confirmed that the first microorganism is a microorganism belonging to a strain different from that of the third microorganism.

(Item 9) In the method for classifying a microorganism according to item 7, the third microorganisms are a plurality of different microorganisms of the order Enterobacterales different from one another at and below one of the family, genus, and species levels, and the obtaining the information includes obtaining information on the classification of the first microorganism based on the first m/z and m/zs of the acid shock proteins in mass spectra of the plurality of different microorganisms.

In the method for classifying a microorganism according to item 9, the first m/z can be compared, at a time, with the plurality of actual m/zs respectively corresponding to the plurality of third microorganisms belonging to different classification categories, and hence the first microorganism can be more efficiently classified as compared with a case where the first m/z is compared with the third m/z corresponding to one of the third microorganisms.

(Item 10) In the method for classifying a microorganism according to any one of items 7 to 9, the obtaining the third m/z includes: obtaining third mass spectrum resulting from mass spectrometry of the third microorganisms belonging to the order Enterobacterales, detecting, as a peak of the acid shock protein, a peak in the third mass spectrum wherein the peak is included in the third mass spectrum, but is not included in mass spectra of the third microorganisms cultured under conditions for not producing the acid shock protein; and detecting a m/z corresponding to the peak of the acid shock protein as the third m/z.

In the method for classifying a microorganism according to item 10, a peak of the acid shock protein can be detected by a simple method.

(Item 11) In the method for classifying a microorganism according to any one of items 1 to 10, the family includes at least one of the family Enterobacteriaceae, the family Erwiniaceae, the family Pectobacteriaceae, the family Yersinia, the family Hafnia, the family Morganellaceae, and the family Budubisiaceae.

In the method for classifying a microorganism according to item 11, the method for classifying a microorganism of the present embodiment can be applied to microorganisms belonging to these families.

(Item 12) In the method for classifying a microorganism according to any one of items 1 to 11, the acid shock protein includes at least a part of at least one of proteins designated as an acid shock protein, an acid-shock protein, an acid shock repeat family protein, an acid shock-inducible periplasmic protein, an acid resistance repetitive basic protein, and a putative acid shock protein.

In the method for classifying a microorganism according to item 12, the method for classifying a microorganism of the present embodiment can be practiced based on at least some peaks of at least one of the proteins designated as above.

(Item 13) In the method for classifying a microorganism according to any one of items 1 to 12, the conditions for producing an acid shock protein include at least one of a condition that involves culturing in a medium supplemented with a sugar, a condition that involves employing an anaerobic state, and a condition that involves performing prolonged culture.

In the method for classifying a microorganism according to item 13, the acid shock protein is produced by culturing the microorganism under at least one of the condition that involves culturing in a medium supplemented with a sugar, the condition that involves employing an anaerobic state, and the condition that involves performing prolonged culture, and thus, the method for classifying a microorganism of the present embodiment can be practiced.

(Item 14) In the method for classifying a microorganism according to any one of items 1 to 13, the mass spectrometry includes at least one of matrix-assisted laser desorption/ionization mass spectrometry and electrospray ionization mass spectrometry.

In the method for classifying a microorganism according to item 14, the method for classifying a microorganism of the present embodiment can be practiced by employing at least one of matrix-assisted laser desorption/ionization mass spectrometry and electrospray ionization mass spectrometry.

(Item 15) A controller that executes classification of a microorganism by mass spectrometry includes a memory; and a processor for executing the method for classifying a microorganism according to any one of items 1 to 14.

(Item 16) An analyzer that executes classification of a microorganism by mass spectrometry includes a detector that measures a mass spectrum of a microorganism; and the controller according to item 15.

It should be regarded that the embodiments disclosed herein are not limiting but illustrative in all points. The scope of the present invention is not limited by the description given above but limited by the scope of appended claims, and is intended to encompass equivalence of the scope of appended claims, and all modifications made within the scope.

### REFERENCE SIGNS LIST

1 Analyzer; 10 Controller; 11 Processing part; 12 Storage part; 13 Input/output part; 20 Detector; 21 Ionization part; 22 Ion acceleration part; 23 Mass separation part; 24 Detection part; 111 Device control part; 112 Mass spectrum creation part; 113 Mass spectrum analysis part; 114 Calibration part; 131 Input part; 132 Output part; 133 Communication part; 221 Acceleration electrode; 231 Flight tube; S Ion.

1. A method for classifying a microorganism, comprising:
obtaining a first mass spectrum resulting from mass spectrometry of a first microorganism belonging to the order Enterobacterales that has been cultured under conditions for producing an acid shock protein, classification of the first microorganism at and below one of the family, genus, and species levels being unknown;
obtaining a first m/z corresponding to the acid shock protein from the first mass spectrum; and
performing classification at or below one of the unknown levels of the first microorganism by analyzing the first m/z.

2. The method for classifying a microorganism according to claim 1,
wherein the performing classification at or below one of the unknown levels includes:
obtaining a second m/z of an acid shock protein produced in one or more second microorganisms of the order Enterobacterales, classification of the second microorganisms at the family, genus, or species level being known; and
performing classification of the first microorganism by comparing the first m/z with the second m/z.

3. The method for classifying a microorganism according to claim 2,
wherein classification categories at all of the family, genus, species, and strain levels of the second microorganisms are known, and
when the first m/z corresponds to the second m/z, the performing classification of the first microorganism includes determining that the first microorganism belongs to the same classification categories at all of the family, genus, species, and strain level as any of the second microorganisms.

4. The method for classifying a microorganism according to claim 2,
wherein the second microorganisms are a plurality of microorganisms of the order Enterobacterales different from one another at and below one of the family, genus, and species levels, and
the performing classification of the first microorganism includes performing the classification of the first microorganism based on the first m/z, and m/zs of acid shock proteins of the plurality of different microorganisms.

5. The method for classifying a microorganism according to claim 1,
wherein the obtaining the first m/z includes:
detecting, as a peak of the acid shock protein, a peak in the first mass spectrum wherein the peak is included in the first mass spectrum, but is not included in a mass spectrum of the first microorganism cultured under conditions for not producing the acid shock protein; and
detecting a m/z corresponding to the peak of the acid shock protein as the first m/z.

6. The method for classifying a microorganism according to claim 2,
wherein the obtaining the second m/z includes:
obtaining an amino acid sequence of the acid shock protein immediately after translation estimated from a gene sequence of the second microorganisms;
estimating protein processing to be performed on the amino acid sequence immediately after the translation;
estimating an amino acid sequence resulting from the protein processing; and
calculating the second m/z based on the amino acid sequence resulting from the protein processing, and
the protein processing includes removal of a signal peptide, and cleavage on a C-terminal side of Gln-Lys-Ala-Gln sequence.

7. The method for classifying a microorganism according to claim 1,
wherein the performing classification at or below one of the unknown levels includes:
obtaining a third m/z of an acid shock protein produced in a third microorganism, the third m/z being obtained by mass spectrometry of the one or more third microorganisms that have been cultured under conditions for producing an acid shock protein wherein the third microorganisms belonging to the order Enterobacterales; and
obtaining information on the classification of the first microorganism by comparing the first m/z with the third m/z.

8. The method for classifying a microorganism according to claim 7,
wherein the obtaining the information includes determining that the first microorganism and the third microorganism are of different strains when the first m/z is different from the third m/z.

9. The method for classifying a microorganism according to claim 7,
wherein the third microorganisms are a plurality of different microorganisms of the order Enterobacterales different from one another at and below one of the family, genus, and species levels, and
the obtaining the information includes obtaining information on the classification of the first microorganism based on the first m/z and m/zs of the acid shock proteins in mass spectra of the plurality of different microorganisms.

10. The method for classifying a microorganism according to claim 7,
wherein the obtaining the third m/z includes:
obtaining third mass spectrum resulting from mass spectrometry of the third microorganisms belonging to the order Enterobacterales;
detecting, as a peak of the acid shock protein, a peak in the third mass spectrum wherein the peak is included in the third mass spectrum, but is not included in mass spectra of the third microorganisms cultured under conditions for not producing the acid shock protein; and
detecting a m/z corresponding to the peak of the acid shock protein as the third m/z.

11. The method for classifying a microorganism according to claim 1, wherein the family includes at least one of the family Enterobacteriaceae, the family Erwiniaceae, the family Pectobacteriaceae, the family Yersinia, the family Hafnia, the family Morganellaceae, and the family Budubisiaceae.

12. The method for classifying a microorganism according to claim 1, wherein the acid shock protein includes at least a part of at least one of proteins designated as an acid shock protein, an acid-shock protein, an acid shock repeat family protein, an acid shock-inducible periplasmic protein, an acid resistance repetitive basic protein, and a putative acid shock protein.

13. The method for classifying a microorganism according to claim 1, wherein the conditions for producing an acid shock protein include at least one of a condition that involves culturing in a medium supplemented with a sugar, a condition that involves employing an anaerobic state, and a condition that involves performing prolonged culture.

14. The method for classifying a microorganism according to claim 1, wherein the mass spectrometry includes at least one of matrix-assisted laser desorption/ionization mass spectrometry and electrospray ionization mass spectrometry.

15. A controller that executes classification of a microorganism by mass spectrometry, comprising:
a memory; and
a processor for executing the method for classifying a microorganism according to claim 1.

16. An analyzer that executes classification of a microorganism by mass spectrometry, comprising:
a detector that measures a mass spectrum of a microorganism; and
the controller according to claim 15.
